(19) United States
(12) Patent Application Publication
(10) Pub. No.: US 2024/0268702 A1
(43) Pub. Date: Aug. 15, 2024

(54) PORTABLE DEVICE ALLOWING ACCURATELY AND CONCISELY CHARACTERISING THE FITNESS CONDITION OF INDIVIDUALS IN ACTIVITY AS WELL AS ACCURATELY CALCULATING AND DETECTING THEIR VENTILATORY THRESHOLDS IN REAL-TIME

(71) Applicant: AGE IMPULSE, Ivry-sur-Seine (FR)

(72) Inventors: Gabriel DIB, Paris (FR); Claire MOLINARI, Sainte-Geneviève-des-Bois (FR)

(21) Appl. No.: 18/562,093

(22) PCT Filed: May 16, 2022
(86) PCT No.: PCT/EP2022/063170

§ 371 Date: Nov. 17, 2023

(30) Foreign Application Priority Data
May 18, 2021 (FR) 2105147

(51) Int. Cl. A61B 5/08; A61B 5/00; A61B 5/0205; A61B 5/024; A61B 5/11; A61B 5/145

(52) U.S. Cl. CPC A61B 5/0816; A61B 5/0002; A61B 5/0205; A61B 5/0823; A61B 5/1112; A61B 5/1117; A61B 5/681; A61B 5/6817; A61B 5/6831; A61B 5/6838; A61B 5/6887; A61B 5/721; A61B 5/7264; A61B 5/7282; A61B 5/7405; A61B 5/742; A61B 5/7455; A61B 5/024; A61B 5/14542; A61B 2560/0242; A61B 2560/045; A61B 2560/0462; A61B 2562/0204; A61B 2562/0219; A61B 2562/0223

(57) ABSTRACT

A portable device for measuring and characterising the fitness condition of a user in activity, including: At least one sensor measuring the respiratory rate and/or at least a second sensor measuring the parameters of the activity of the user; Means for accurately calculating concise parameters that characterise the fitness conditions, including the VO2max, and for calculating the ventilatory threshold in real-time; Means for characterising the activity of the user; An interface for interaction with the user; the device implementing an algorithm enabling an analysis of the measurement parameters and the calculations.

### CROSS REFERENCE TO RELATED APPLICATIONS

This application is a National Stage of International Application No. PCT/EP2022/063170, having an International Filing Date of 16 May 2022, which designated the United States of America, and which International Application was published under PCT Article 21(2) as WO Publication No. 2022/243235 A1, which claims priority from and the benefit of French Patent Application No. 2105147 filed on 18 May 2021, the disclosures of which are incorporated herein by reference in their entireties.

### BACKGROUND

### Field

The present disclosure relates to a portable device allowing characterising accurately and concisely the physical fitness condition (circulatory, respiratory and locomotor systems) of individuals in activity (for example walking, running, cycling, rowing, exercising on an elliptical trainer, practising a rhythmed exercise), as well as accurately calculating/detecting their Ventilatory Thresholds thereby allowing adapting the activity to the physical fitness level “of the moment”.

The device according to the present disclosure is based more particularly on the calculation of V02max, while improving the accuracy of this calculation thanks to the Respiratory Rate, measured in a manner suited to the activity, resistant to artefacts and also in real-time. The measurement of the Respiratory Rate also allows accurately calculating/detecting the Ventilatory Thresholds.

### BRIEF DESCRIPTION OF RELATED DEVELOPMENTS

With the advent of many mobile applications, with or without portable devices (“wearable”), the users are able to simply monitor their individual health-related data throughout the day, such as the number of travelled steps, the consumed calories, the heart rate, etc., as well as other measurements of their personal activity, such as the quality of sleep. Nonetheless, they lack a concise and accurate indication on their fitness condition and its evolution, which is accessible to the greatest number.

Thanks to the portable device, the present disclosure, allows accurately characterising the fitness condition of the person, in particular through the calculation of the V02max and of the integration of the measurement of the respiratory rate in this calculation. V02max is the scientific, concise and most accurate indicator of the physical condition and encompasses the circulatory, respiratory and locomotor systems. The method has been developed with respect to the values of V02max measured in the laboratory and validated with different exercise modes. When applied to our disclosure, the accuracy of the method is 95% (absolute mean error in percentage, MAPE ˜5%). The error is similar to that of the method referenced by professionals in the laboratory. It is difficult to do better for a physiological measurement.

VO2 Max is an indicator well-known to sportsmen. It is an excellent measurement of the physiological age and is closely correlated with the active life expectancy. V02max is expressed in litre of oxygen per minute (L·min−1), however, in order to take account of the different morphologies, its value is divided by the weight. It will then be expressed in ml·kg−1·min−1.

It is assumed that the higher its value, the more the physiological age will be young. Because a high V02max means that the body is more able to absorb oxygen, to convey it to the muscles, and to be transformed to create the energy fuel that the muscles consume to contract and function. It is important because this energy source is one amongst the most effective ones for the body.

When talking about advanced age, one think about a decrease in V02max and physical capabilities, accentuated by a sedentary lifestyle. Nonetheless, by adopting physical or sports activity practice habits, the fitness condition improves and it is possible to “reverse the aging curve”.

Thus, our characterisation of the fitness, including V02max, allows recommending customised and evolving exercises thanks to a decision engine. This customisation is enhanced by the adaptation of someone's exercises to the perception of the effort of the user (Easy, Average, Intense), favouring listening to oneself, thereby avoiding the risk of an accident or a feeling of failure.

The method for measuring V02max, referenced by professionals, is very accurate, but requires professional equipment that is expensive and complex to use, and also requires means in terms of time and trained resources for the calibration of the equipment and the interpretation of the results.

The ergometer is known in the prior art, with a mask connected to fixed equipment, in order to continuously monitor the content and the flow rate of the respiratory gases. This allows assessing the maximum oxygen absorption (V02max) of the subject and his/her cardio-respiratory endurance.

The Cosmed (registered trademark) K5 device, which is composed of a mask connected to an electronic unit to be carried like a backpack, is also known in the prior art, which allows performing the laboratory test in a mobile manner. This unit is a portable gas exchange analyser. Associated with this device, there are a chest belt, a heart rate monitor, a sensor (inertial platform) allowing assessing walking/running parameters and a GPS watch, an activity tracker.

During the usual method referenced by the professionals to carry out this test in the laboratory, the subject performs an exercise until exhaustion. This exercise can be qualified as extreme and not recommended for fragile seniors.

Moreover, simpler and field-deployable methods for the measurement of V02max could be considered in a 6-minute walking test, a 12 minute Cooper's run test, or a fixed-distance run test, etc. Under these conditions, the matter is to assess the travelled distance and the maximum speed maintained. However, these methods are not very accurate, in particular because it is difficult to maintain a constant and maximum speed for more than 8 minutes. Indeed, the maximum aerobic speed that enables these tests to estimate V02max could be sustained only over a duration of 2 to 8 min These simple test methods reflect the level of cardio-respiratory endurance of a subject but the accuracy of the result is lower than a laboratory method.

During a so-called simple test as described hereinabove (walking for 6 minutes, Cooper for 12 minutes) or else during assessments based (in particular in smartwatches) on the heart rate and the speed, the typical error is at least 10-15%.

Moreover, another simple and field-deployable method for measuring the V02max uses, via a Mobile Application, the inertial platform of the Smartphone or of the smartwatch without the respiratory rate (RR). The accuracy of the result is lower than a laboratory method.

The ventilatory threshold (VT) corresponds to a major physiological change: the energy production mode changes from an (aerobic) oxygen consumption mode to an anaerobic mode. This transition is called the anaerobic threshold. Exercising at or above the transition is useful for sportsmen but is not possible for a long time and is not recommended for fragile persons. In the prior art, the measurement of the heart rate (HR) is used in the physical and sports training to estimate the respiratory rate and the ventilatory threshold. The usual training protocols are based on two approximations: 1) the HR max (the greatest number of heart beats per minute that an individual can reasonably reach in an intense exercise, estimated by different formulas. Yet, it has been demonstrated that taking into account the effect of the age on the HR with regards to the equation for determining the theoretical maximum HR does not always provide reliable results) 2) a percentage of this HR max, between 70 and 90% depending on the condition of the person.

The respiratory rate is still badly recorded in the healthcare field, despite substantial evidence of its clinical relevance. The respiratory rate (RR) is a fundamental vital sign which is sensitive to different pathological conditions (for example, adverse cardiac events, pneumonia) and stress factors, in particular emotional stress, cognitive load, heat, cold, physical effort and fatigue induced by the exercise.

The sensitivity of the respiratory rate to these conditions is higher than that of most of the other vital signs, and the possibility with our device to measure the respiratory rate has major implications for the healthcare field, professional environments, and sport.

Similar values of RR could be observed when the user is subjected to a cognitive load, an emotional stress, a pain, a dyspnoea or simply by a moderate exercise.

This problem could be partly overcome by the simultaneous measurement of other physiological, mechanical and environmental variables (for example temperature, noise). For example, it is important to characterise the postures and the activities of the user, as they are carried out with the use of the inertial sensors of the device.

The respiratory rate (values expressed both in breaths/min and Hz) could change in response to different factors.

A high RR at rest has been found to be the most accurate vital sign to predict a cardiac arrest compared to the heart rate and the blood pressure in hospitalised patients. The increase in the RR at rest is observed a few hours before the occurrence of a cardiac arrest, thereby suggesting that monitoring of the RR could assist in the early detection and management of undesirable cardiac events.

The importance of these results is not restricted to healthcare facilities, but extends to home monitoring of patients at risk, since the cardiac arrest outside the hospital is one of the main causes of cardiac death in the world. Thus, the respiratory monitoring could assist in the prediction or early management of such an event.

Recently, free aptitude test modes have also been introduced in EP0709058 (Alessandri), U.S. Pat. No. 6,882,955 (Ohlenbusch & Darley), FR2867055 (Quilliet & Vortex), US2007/0082789 (Nissila, Niva, Jaatinen & Kinnunen), and by Weyand et al. (2001)). These tests combine the measurement of the heart rate and the speed during the exercise performed by the user, where the maximum oxygen absorption is determined, for example, using simple mathematical calculations. The speed may be measured using an accelerometer and/or using one or more satellite positioning system(s) (for example the GPS).

Physicians pass examinations whether for sports or rehabilitation (cardiological, etc.) purposes on an ergometer (treadmill, bicycle, rower trainer, etc.) by gradually increasing the intensity of the effort until the person reaches a heart rate (HR) at least higher than 70% of his/her theoretical maximum heart rate (estimated and not measured). Based on this examination, they derive a generally linear relationship between the speed (power) and the HR.

While the heart rate continues to increase linearly with the speed, the respiratory rate (RR) increases in an exponential manner that corresponds to the Ventilatory Threshold (VT), a moment in a effort at which the latter becomes “hard”.

It is quite easy to identify the threshold of this sudden arduousness by the hyperventilation threshold (ventilatory threshold) while the HR does not modify its linear increase and does not allow detecting this threshold.

In the prior art, some other devices measure the change in variability of the HR with the effort, since this change in variability is the sign of the effect of stretching of the sinus node by the muscles of the ventilation at ventilatory threshold time. This determination implies a high accuracy of the heart variability thanks to indexing of each heartbeat, which, with effort, with artefacts, and especially in seniors, is difficult (noise/signal ratio) except when the apparatus for detecting each beat is sophisticated and consequently very expensive.

The document U.S. Pat. No. 5,810,722 assigned to the Polar Electro company describes a device for assessing the VT of a person under progressively increasing stress. The respiratory rate and volume are calculated on the basis of the electrocardiogram (ECG) signals to exploit a respiratory rate graph as a function of the heart rate, or a ventilation vs. heart rate graph, where the VT appears as a breaking point. A difficulty related to this method is that it is entirely based on ECG signals. Indeed, the determination of the respiratory response based on the ECG, although theoretically possible, requires a high-quality signal, which is not always compatible with the field measurements.

In the prior art, the measurement of the Respiratory Rate is also performed by different other methods based on measurement(s) of other physiological parameter(s) such as a blood flow or a movement of the thoracic cage. Nonetheless, these solutions are either not very accurate, or intrusive and not very suited for example to exercises incorporated in the daily life of seniors.

The European patent No. EP 0 809 965 B1 (Seiko Epson Corporation) is also known in the prior art, which relates to a health condition monitoring device and an exercise assistance device.

The European patent No. EP 2 059 166 B1 (Fresenius Medical Care Deutschland GmbH) is also known in the prior art, which relates to a method and a device for determining the respiratory rate.

The European patent No. EP 2 773 263 B1 (LifeLens Technologies) is also known in the prior art, which relates to a metabolic and cardio-pulmonary monitoring device.

### SUMMARY

The present disclosure provides a portable device allowing accurately characterising the physical fitness condition of a user in activity, in particular by taking into account the parameter V02max and by improving the accuracy of this calculation thanks to the respiratory rate, measured in a manner suited to the activity, resistant to artefacts and in real-time. Thanks to the measurement of the Respiratory Rate, the device also allows calculating and accurately detecting the ventilatory threshold.

The method and system for characterising the fitness and for calculating in particular the V02max according to the present disclosure address the defects of the prior art. The data relating to the respiratory rate and to the biomechanical parameters of the activity are acquired by a smart portable device. The maximum oxygen consumption is calculated using the method provided by the disclosure, and the measurement of the respiratory endurance is carried out in a practical and rapid manner. In addition, the method according to the present disclosure avoids the user being stressed due to wearing of the mask for the analysis of the gas exchanges, the costs of the laboratory test, the time required for the calibration and wearing of the equipment, the interpretation of the results and also exhaustion due to conventional test methods.

Thanks to the portable device, the present disclosure also allows measuring and detecting (and not estimating) the ventilatory threshold (VT). It is an important physiological parameter for training sportsmen but also seniors. The VT allows adapting the activity to the physical fitness of the day of the athlete, of the sportsman, of the young or of the senior, to overcome the risk of overtraining and recurrent exhaustion if the athlete is not at his/her usual physical level (disease, dehydration, stress, etc.), or to avoid a health accident to the senior person.

Users can monitor their VT on a daily basis, without going to a laboratory. This is of great interest in the development of their training programs. Indeed, the determination of the VT during and/or directly after the physical effort allows for a better improvement of the endurance and therefore of the V02max.

The present disclosure aims to provide a solution accessible to the greatest number, by making a laboratory method that requires expensive equipment, time and trained resources affordable. The solution is based on 30 years of scientific and field experience, 140+ international scientific publications, validated on athletes, young and senior persons, including Robert Marchand, an amateur cyclist, World champion at 105 years, who had a V02max of a 50-year-old person. This accessibility is accentuated by the daily integration of customised and evolutive exercises (for example 3 times 30 minutes per week of walking or running, stair climbing) and by the cost of the solution. This customisation is enhanced by the adaptation of these exercises to the perception of the effort by the user (Easy, Average, Intense), favouring listening to oneself, thereby avoiding the risk of an accident or a feeling of failure.

To this end, the disclosure relates to a portable device allowing measuring and characterising the physical fitness condition of a user in activity, including:
At least one sensor measuring the respiratory rate and/or at least one second sensor measuring parameters of the activity of the user;
Means for accurately calculating synthetic parameters which characterise the fitness including the V02max and for calculating the ventilatory threshold in real-time;
Means for characterising the activity of the user;
An interface for interaction with said user;
   said device implementing an algorithm enabling an analysis of the measurement parameters and the calculations.

The present disclosure allows inciting sedentary persons who do not know how to return to activity without the fear of falling again into the discomfort of the physical activities which would not take into account the current level of their physical and mental fitness.

Athletes, sportsmen as well as young and senior persons can also be motivated to achieve an objective, to initiate a preventive and/or corrective action to improve their physical fitness or other well-being criteria and to reduce chronic disease risk or fall risk factors. Physical inactivity and bad physical condition are associated with several health problems, such as cardiovascular diseases, metabolic disorders (for example, overweight, obesity, diabetes), musculoskeletal disorders, lung diseases, etc. It has been demonstrated that the improvement of the cardio-respiratory endurance, i.e. of the physical fitness condition, reduces mortality, all causes combined. In practice, for a person in bad physical condition, an increase in 10% of the V02max could reduce the risk of mortality by 15% and give him/her 10 additional years of good life quality, as illustrated in FIG. 1.

Thanks to the device according to the present disclosure, the users can monitor their ventilatory threshold on a daily basis, without any laboratory effort. This has a great interest in the context of the development of their training programs. Indeed, the determination of the ventilatory threshold during and/or directly after the physical effort allows for a better training, an improvement in endurance and therefore of the V02max.

Another specificity of the device according to the present disclosure is to give training instructions to the user based on the perception of the effort. This allows listening to oneself and not following trainings based on performance criteria, not customised (for example following a speed, heart rate setpoint) with the risks of injury or exhaustion.

Thus, the user is capable of associating an effort perception setpoint with suited cardio-respiratory resources without this leading to constraint, allowing him/her to better adapt the training to his/her physical condition; this training having been already customised following the performed balance and the characterisation of his/her fitness.

Usually, a cardio-respiratory assessment is done in the laboratory or in the field with fixed speed setpoints, which increase at given time points, until the person reaches exhaustion.

In fact, replacing the perception of the effort by the user as a major influence factor of his/her evolution allows making the performances progress without reaching the limits.

This method based on the perception of the force will enable the user to understand that the improvement of his/her physical condition is not a permanent struggle against the barrier of the cardio-respiratory limits but an understanding and an adaptation of the governance of his/her fitness condition.

The present disclosure takes advantage of other accurate measurements of the portable device. Thanks to the integrated inertial platform (3D accelerometer, 3D gyroscope, magnetometer, etc.), measurements of the walk/gait parameters allow measuring the risk of fall and cognitive impairment based on the clinically proven results of different scientific publications. The accuracy of the measurements is considerably improved thanks to the possibility of positioning the portable device at the bottom of the back, close to the centre of gravity of the body.

As regards falls, for example, each year in France, 20 to 30% of the more than 65-year-old persons, and 50% of the more than 85-year-old persons are victims of one fall at least. 15% of the falls are responsible for bone trauma (femoral neck fracture in 30% of cases).

The inability for the elderly person to raise is a bad prognosis. Staying on the ground for one hour is a gravity factor with a risk of death in 50% of cases in the next 12 months.

In this context and with the mass arrival of the babyboomers, the prevention of falls could provide a response to a human and public health concern:

Reduce the number of falls and decrease their cost evaluated to more than 2 Mds € in France, $50 Mds in the United States
Increase the life expectancy without inability, and reduce the number of deaths (more than 12,000 a year in France, 27,000 in the US)

As regards neurodegenerative pathologies, the concern is to screen Mild Cognitive Deficits exposing the risk of dementia at the earliest.

The accurate measurements of the walking/gait parameters also allow practicing Feedback Training by training and monitoring, in real-time, more particularly a parameter that deviates.

According to one aspect, said device is autonomous.

According to another aspect, said device is connected to another device of the smartphone, smartwatch, digital tablet type which are connected to the Cloud or said device is connected directly to the Cloud.

According to one implementation, said interactive interface is a graphical interface.

According to another implementation, said interactive interface is an audio interface.

Advantageously, said device is associated with an accessory, allowing clipping it, or fastening it or integrating it into a piece of equipment or inserting it into a piece of equipment.

According to one aspect, the algorithm is implemented in real-time.

According to one aspect, the algorithm is embedded in the device.

According to another aspect, the algorithm is executed partially or totally outside the device.

According to one aspect, said device includes at least one microphone.

Advantageously, the at least one microphone is unidirectional.

Advantageously, said device includes several omnidirectional microphones, placed in an array (beamforming), with a signal processing to create a directional microphone, which allows placing the device away from the mouth/nose.

Advantageously, said device implements an algorithm for detecting audio rhythms or artificial intelligence (for example Deep Learning) of the microphone(s) in order to extract therefrom the Respiratory Rate in a manner suited to the activity, resistant to artefacts, in a noisy environment.

Advantageously, said device includes redundant sensors (for example redundant microphone(s), or microphone(s) and probe for detecting the change in temperature due to breathing) in order to make the measurements more reliable.

Advantageously, said device includes an inertial platform which includes a 3D accelerometer sensor, a 3D gyroscope, a magnetometer, a barometer and/or a satellite positioning system.

Advantageously, said device further includes a microcontroller or an artificial intelligence processor for IoT devices, a memory, a BLE or other transmission mode module, a battery, one or more LED(s), one or more LED(s), a multifunction button (for example for activation, recording, transmitting and deleting the records, reset) or a plurality of buttons, a loudspeaker, a buzzer and/or vibrator, as well as other components of an IoT system.

For example, these components of an IoT system may consist of a battery with or without a capacitor, a touchscreen and/or a tactile interface, a wired socket, a system for charging the battery by induction, an audio jack and/or a voice control system.

According to one aspect, said device further includes a body parameter sensor and/or an environment parameter sensor.

According to one aspect, said device further includes means for detecting an alert.

According to one aspect, further comprises means for analysing and characterising the breath and/or the cough via an artificial intelligence module.

Advantageously, said device produces training instructions based on the perception of the effort and/or the ventilatory threshold.

Advantageously, said device includes means for preventing the risk of falling and for carrying out Feedback Training by training and monitoring a defective parameter.

According to one aspect, the sensor also measures the actimetry of the user.

The present disclosure also relates to a system including the above-mentioned device measuring in particular the respiratory rate, as well as at least one 2nd device, with an inertial platform allowing measuring the rhythm of a cyclist, rower, etc., and/or parameters of the activity, the first device clipped at the jacket or T-shirt, cannot measure, for example, the rate of a cyclist or a rower.

Thanks to this modular approach, at least one additional device including complementary sensors would allow performing other measurements.

The present disclosure also relates to a system including the above-mentioned device, a device of the smartphone, smartwatch or digital tablet type, and a cloud-based server.

Advantageously, said system further includes at least one second measuring device for measuring the rhythm for rhythmic activities or for performing other measurements.

According to one aspect, said system further includes another device coupled with the first device. Indeed, when running or walking, the same device can measure the respiratory rate and the rhythm/speed. To measure a rhythm in addition to the respiratory rate when cycling, when exercising on an elliptical trainer or when rowing, it is necessary to have this second device which could be placed on the wrist or on the handle of the rowing machine, on the foot or on the elliptical machine/the pedal of the bicycle. This second device may also be positioned at another location of the body, for example the bottom of the back, in particular to measure synchronisation with the top of the body when exercising or walking.

### BRIEF DESCRIPTION OF THE DRAWINGS

FIG. 1 shows the physical fitness as a function of age

FIG. 2 shows a device according to the present disclosure, in one aspect.

FIG. 3 illustrates the architecture of the system implementing the device according to the present disclosure.

FIG. 4 shows the signal flow processing.

FIG. 5 illustrates a conceptual view of an audio noise reducer.

FIG. 6 shows a noise reduction example.

FIG. 7 illustrates the detection of the envelope.

FIG. 8 shows the rhythm extraction flow.

FIG. 9 illustrates the signal flow based on machine learning.

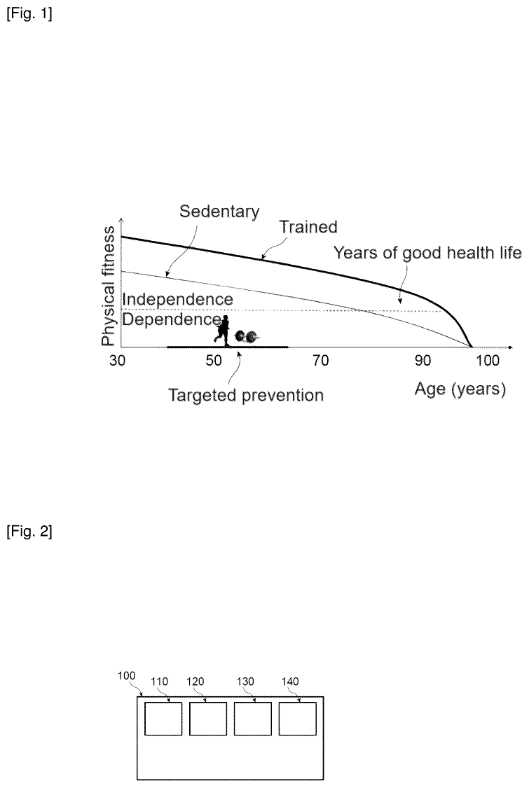

### DETAILED DESCRIPTION

The present disclosure relates to a portable device 100 allowing measuring and characterising the physical fitness condition of a user in activity, including:
At least one sensor 110 measuring the respiratory rate and/or at least one sensor 140 measuring parameters of the activity of the user;
Means 120 for accurately calculating synthetic parameters that characterise the fitness including the V02max and for calculating the ventilatory threshold in real-time;
Means for characterising the activity of the user;
An interface 130 for interacting with said user.

The device 100 further implements an algorithm enabling an analysis of the measurement parameters and the calculations.

The measurement of the respiratory rate (and not an estimation based on the heart rate as commonly practised), in addition to the activity sensors of the inertial platform, adds in significant accuracy to the calculation of the V02max (we reach the 5% error margin of the physiological measurements).

The accurate measurement of the respiratory rate in real-time also allows accurately detecting the ventilatory threshold (VT). This threshold is important for training sportsmen (senior or not) but it is nowadays estimated very inaccurately. The ventilatory threshold accurately detected, thanks to the measurement of the respiratory rate, is also useful for seniors in order to avoid accidents during exercise sessions.

The device 100, including in particular the sensors 110 and 140, is non-intrusive, and is positioned for example at the collar of the shirt/T-shirt, or in a headband, a hairband or else integrated with earphones, which thanks to:
beamforming created by inexpensive omnidirectional MEMS microphones can create a directional microphone to remotely capture the respiratory rate at the nose/mouth (without having to position a directional microphone in front of the mouth).
software for processing the audio rhythm or AI software (Deep Learning) can measure the respiratory rate, in the audio signal supplied by the beamforming, in a noisy environment;
an inertial platform and the associated software, to analyse the parameters of the activity, for example the walk, the run and the gait.

The sensor 140 measures parameters of the activity of the user.

In addition, with a simple microphone, the device, positioned in front of the mouth, allows accurately measuring the respiratory rate, parameters of the walk, brings in a significant accuracy in the calculation of the V02max and also allows accurately detecting the ventilatory threshold.

In one aspect, the device 100 includes several omnidirectional microphones, placed in an array (beamforming), with a signal processing to create a directional microphone, which allows placing the device away from the mouth/nose.

In one aspect, the device 100 implements an algorithm for processing the audio of the microphone(s) in order to extract therefrom the respiratory rate in a manner suited to the activity, resistant to artefacts, in a noisy environment.

In one aspect, the device 100 includes redundant sensors to make the measurements more reliable.

FIG. 2 shows a device 100 according to the present disclosure, in one aspect.

The device 100 according to the present disclosure is portable, miniaturised, non-intrusive, non-stigmatising and has a great autonomy.

It allows accurately measuring and monitoring the physical fitness condition of an individual in activity (for example: walking, running, rhythmed physical exercise), its ventilatory threshold and guiding his/her training. A second device allows measuring the exercise rhythm such as rowing, cycling, training exercising an elliptical trainer, etc.

In one aspect, the device 100 according to the present disclosure is autonomous.

In another aspect, the device 100 according to the present disclosure is connected to another device 200 of the smartphone, smartwatch or digital tablet type, which are connected to a “Cloud”. The device 100 may also be directly connected to a “Cloud”.

The device 200 of the smartphone, smartwatch or digital tablet type can take the initiative to activate the device (for example for the Balances, exercise sessions or the fall risk assessment test). The device 200 may be on standby for the rest of the time. The device 100 may also take the initiative of activation when the user activates it, for example using a button placed thereon or by moving it.

The device 100 according to the present disclosure implements an algorithm enabling an analysis of the measurement parameters, in real-time or not, embedded in the device or not and an interactive application 130.

This algorithm is embedded in the device, or is executed partially or totally outside the device.

In one aspect, the interactive interface 130 is a graphical interface.

In another aspect, the interactive interface 130 is an audio interface.

The interactive application 130 gives instructions and advice regarding how to adapt someone's physical activity in order to achieve the customised fitness maintenance or recovery objectives.

In one aspect, the sensor further measures the actimetry of the user.

In one aspect, the device 100 according to the present disclosure is associated with an accessory, allowing clipping it or fastening it to integrate it into a piece of equipment or to insert it into a piece of equipment. For example, it may be clipped with a clothespin-type accessory on the collar of a jacket or on a T-shirt to measure the respiratory rate and the parameters of the walk. It may also be clipped at the base of the back close to the centre of gravity, on shorts, a belt, trousers, a skirt or a tracksuit, in order to accurately measure the parameters of the walk. The device 100 according to the present disclosure may also be inserted into a pouch of a briefs, for actimetry. It may also be affixed on a mask, of the anti-virus or anti-pollution type. This allows measuring the respiratory rate, even when wearing the mask. For example, the device 100 according to the present disclosure may also be clipped onto laces of a shoe, fastened on the trousers or the crankset to measure the cycling rhythm or be integrated into an anti-sweat wrist band or attached on the grip of the piece of equipment of the rower trainer type to measure the rhythm. Finally, the device 100 according to the present disclosure may also be integrated in a headband or in earphones or in “earpods” type apparatuses.

The originality also lies in the possibility of using the device 100 according to the present disclosure for the accurate analysis of the parameters of the walk, such as the speed, the rhythm, the regularity, the craniocaudal power, when it is positioned at the bottom of the back, close to the centre of gravity and thus enable the assessment of the risk of fall. A simple test protocol has been retained, following scientific publications and field studies. It is also possible to use the device 100 according to the present disclosure, not necessarily positioned at the bottom of the back, to analyse the actimetry of the user.

The analysis of the walk parameters in real-time, for example on a walking belt in kinesitherapy facility and/or in a sports club and/or when walking or running outdoors, allows monitoring a parameter and improvement thereof in real-time, thanks to the advice of the physiotherapist. This enables a stimulating and game-like interaction for the therapy of gait disorders. The parameters may be: speed, stride length, rhythm, unbalance, regularity, symmetry, pathogenic shocks, total power.

The device 100 according to the present disclosure also allows detecting an alert, for example if the user requests assistance or if the device detects an emergency (an event detected for example by the microphones), or if he/she falls (an event detected for example by the inertial platform).

The analysis of the breath and/or of the cough by AI also allows remotely monitoring a pathology following the administration of a treatment, or detecting an alert following the degradation of the health condition, for example for patients with COPD (chronic obstructive pulmonary disease), detecting cardiac disorders, sleep apnoea, pneumonia, dyspnoea, stress, intellectual load, etc.

Indeed, the objective measurement of the frequency, its intensity as well as this AI analysis of the sound signature and of the duration of the associated noises such as rhonchi, sibilances, crackling, pleural effusion, cough, sneezes, etc., contain important information for physicians during remote monitoring, for example of drug efficacy, also during respiratory functional explorations, and rehabilitation exercises, in particular, when these exercises and explorations are carried out by technicians in the absence of the physician, etc.

These measurements also allow identifying the moments and the contexts of the triggers as well as the triggering factors. The identification and the analysis of the triggers related, for example, to professional diseases on the workplace, or to an infectious disease or else to a pollution problem in a geographical area, allow alerting in particular the healthcare stakeholders.

These measurements may also be exploited for clinical, research, statistical purposes, etc. AI also allows clinical teaching.

The solution is modular:

One or more other device(s) allow(s) measuring the parameters of some activities for example the cycling or rowing rhythm, in addition to the device for measuring the respiratory rate. These may also integrate other types of sensors (ECG, SP02, etc.)

Several sensors may be added, in the same device 100 according to the present disclosure or in a different device, to validate the same measurement, for example of the respiratory rate with a double measurement, of the same audio sensor type or of two sensors of different nature for example an audio sensor, a mini temperature probe (which detects the change in temperature following breathing), or with a mini C02 probe whose response varies with breathing, or moisture or the like. The fact that the sensors are of different nature does not make them sensitive to the same artefacts. A measurement of a predefined deviation of the new measurement with respect to the preceding one allows retaining one measurement rather than another which would have been subject to an artefact.

The audio interaction between the user and the application is done via the loudspeaker of the smartphone or the personal earphones, or via his/her hearing aid, connected to the application of the smartphone (or to the application of the smartwatch or of the digital tablet) or else directly to the device 100.

The guidance of a training session may also be carried out by the device 100 according to the present disclosure without using the application (for example by the audio of the device). Loading of the session in the device 100 according to the present disclosure is done by the application.

An implementation of the solution may be considered in OEMs in an earpiece with an integrated microphone which also captures the respiratory rate and with an inertial platform which measures the parameters of the walk.

The device 100 according to the present disclosure can operate autonomously without going through the smartphone or the cloud. Thus, it can detect the ventilatory threshold in real-time and signal it to the user (for example via a buzzer).

Different aspects of the present disclosure are described hereinbelow:

Intrinsic directional microphones, like cardioids, are a good theoretical solution to maximise a specific sound recording, by eliminating many undesirable external noises by the microphone device itself.

With several omnidirectional microphones placed in a network, with an additional processing of the signal, it is possible to recreate the directional pattern. Depending on the number and size of the array, the main direction may be modified in the direction and in the aperture angle and may vary with the frequency. This could be a good solution to optimise recording of a specific sound.

With only two omnidirectional microphones, it is possible to create a given directivity pattern on the main axis created by the 2 diaphragms using a beamforming audio processing system. This system is nowadays used, for example, on many earphones to accentuate the sound originating from the mouth for telephone calls.

Different options are available for applying a digital audio processing to a sound record. We consider three modules:
The recording apparatuses (integrated in the device 100 according to the present disclosure)
The smartphone, smartwatch or tablet 200
The server 300 in the Cloud

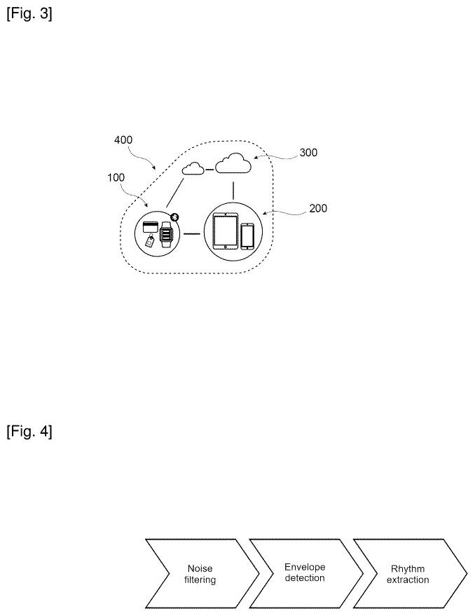

FIG. 3 shows the architecture of the system implementing the device 100 according to the present disclosure. This figure shows the device 100 according to the present disclosure, another device 200 of the smartphone, digital tablet or smartwatch type and a cloud-based server 300.

Depending on the overall constraints of the system, the filter of the respiratory rate and the processing of the artificial intelligence or of the audio rhythm and the analysis of the parameters of the activity, for example walking or running, may be integrated at different locations.

The choice depends on:
The load of the CPU: expressed in MIPS (million instructions per second) or in MHz. It provides information on the complexity of a software. The processor must take on more MIPS or MHz than is required by the algorithm to ensure real-time capabilities.
The load of the AI component (“AI at the edge”).
The memory requirements: expressed in kB or MB (kilo-bytes and mega-bytes). There are three different memory types:
   the program memory, which stores the executable software,
   a static memory, which stores the parameters and the filtering coefficients,
   the dynamic memory, which is used by the processing as a temporary space.
The latency constraints: expressed in seconds. It is the response time between the recording of an event (human breathing) and the availability of the value.

Conventionally, high-stress functions in real-time, such as noise reduction and echo cancellation for the communication voice, are integrated into the recording device (in Bluetooth earphones for example).

When a storage memory is large (ratios, diagnostics) and greater processing capacities are necessary, the smartphone is preferred. The cloud processing is an option for the artificial intelligence processing and the data exchange/protection.

Nonetheless, embedded artificial intelligence processing (AI at the Edge) are nowadays available with high performances and a low consumption.

Next, we will describe the audio processing flow, firstly in a simple approach, then in an advanced approach.

FIG. 4 shows the signal flow processing.

Firstly, an adaptive LMS noise filter attempts to reject the disruptive signals. This filter allows eliminating all continuous/stationary background noises such as the engine of the car, the air-conditioner, etc.

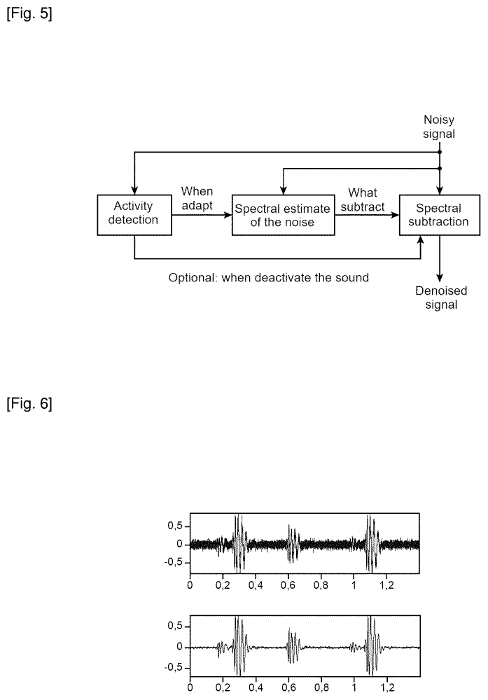

FIG. 5 illustrates a conceptual view of an audio noise reducer.

In the frequency domain, the algorithm identifies the characteristics of the noise and detects the dynamic activity of the signal to be preserved. Afterwards, a spectral subtraction is applied between the original sound and the estimated noise. It is not possible to suppress more than 10 dB of noise without creating significant artefacts. The system should be accurately adjusted to obtain the best balance between noise and the presence of artefacts.

FIG. 6 shows a noise reduction example.

The second step is an envelope detector. This algorithm smoothens the waveform to recover the average shape of the signal. The filter based on a low-pass filter also reduces the undesirable residual noise.

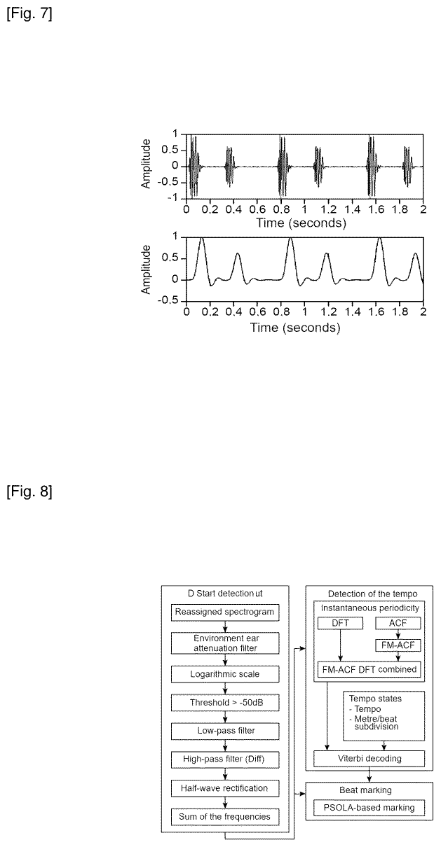

FIG. 7 illustrates the detection of the envelope.

The last step, through the analysis of the shape of the envelope, extracts the time-stamps from the local maxima. Thus, the respiratory rate can be easily extracted.

This approach allows understanding the different modules that are studied to detect breathing based on the microphonic records.

Advanced methods are described hereinafter for detecting the respiratory rate based on an audio record which improve the quality of the result, in particular in noisy environments.

In the current context, the audio record comprises both strong noises (movements of human clothes, environment) and the sound of human breath. A robust rhythm detection algorithm should be used to guarantee the best performances.

A large number of publications have been developed during the last two decades, and each year, many requests are submitted to Music Information Retrieval Evaluation exchange (MIREX). It is possible to consider that the respiratory rate detector is similar to the assessment of the music beats.

A common general strategy for rhythm monitoring operates in two steps. Firstly, the audio signal is processed by an apparition force function, which measures the probability that a major musical change (for example, the apparition of a note) is produced at each time point. Afterwards, the monitoring algorithm selects the beat times from among the peaks of the profile of the attack force.

The apparition energy function is first proposed based on the reassigned spectral energy flow. A combination of the discrete Fourier transform and of the mapped frequency autocorrelation function is used to estimate the dominant periodicities at each time. Afterwards, a Viterbi algorithm is used in order to detect the most likely subdivision trajectories in terms of tempo and beats over time.

FIG. 8 shows the rhythm extraction flow.

In the audio domain, the artificial intelligence model gives better results than the conventional filter based on an algorithm. The following approach combines the noise reduction and the rhythm extraction into one single artificial intelligence system.

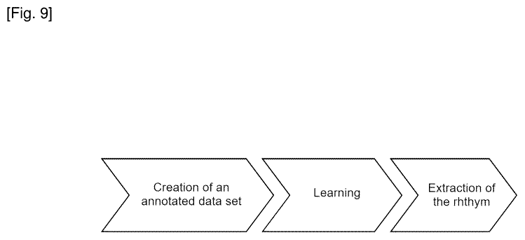

FIG. 9 illustrates the signal flow based on machine learning.

First, there must be a set of reference audio data. It may consist of a real recording, but this may be a very long task when numerous noise levels and environments are necessary. An alternative consists in using the existing noise databases on the one hand and a clean/annotated record, with the actual rhythm, of breathing on the other hand.

The database may then be created artificially by mixing the noise and the clean signal with different weights. Thus, the variations may be infinite.

Different databases on the noise are available.

An inertial platform with a very small footprint may be integrated into the device 100 according to the present disclosure

In one aspect, the device 100 further includes a body parameter sensor and/or an environment parameter sensor. It may also include several body parameter sensors and/or several environment parameter sensors. For example, one or more microphone(s) for analysing the sounds, noises or sound pollution of the streets or places visited by the user, at a given time, a day of the week or of the month, which would allow informing and sharing this information.

This could consist of the measurement of the outside temperature or that of the body, the heart rate, the oxygen saturation level (Sp02). These measurements of the parameters of the body may be performed thanks to the device integrated into earphones.

Another example would be pollution sensors in the streets or places visited by the user, at a given time, a day of the week or of the month, which would also allow informing and sharing this information.

The present method allows accurately detecting and measuring the ventilatory threshold (VT) by the respiratory rate. Yet, with this technological lock that we have overcome, we already have a scientifically validated protocol allowing, thanks to this detection of the VT, developing a highly customised training protocol that is articulated around this threshold. This protocol allows defining accurately, individually, in real-time, the effective durations and intensities of the trainings.

In order to determine the ventilatory thresholds, the user carries out a test, for example a walking or running test, in the field. The test may be self-administered, requiring no special test equipment or trained staff.

The present disclosure suggests two methods for determining a breaking point in a physiological data set, a method carried out at the end of the session, for data acquired during the exercise, the other method being carried out during the session, for data acquired in real-time.

The two methods comprise a preliminary filtering step, in which the physiological data are processed in order to eliminate the data corresponding to recovery or stability periods during the exercise session and to keep only increasing trends as selected data. Hence, the resulting data represent an incremental effort, and the ventilatory threshold is determined based on these data as the critical point at which the ventilation starts to increase more rapidly.

The first method, at the end of the session, analyses the obtained information, once the user confirms that the test is completed. Afterwards, said method comprises, in a possible aspect, a step of identifying two lines with a different slope which correspond to data selected from the set of physiological data, wherein said breaking point corresponds to the intersection of said lines.

This first method may also comprise, in another possible aspect, the calculation of the second derivative of the best-suited polynomial function and the detection of the extrema that denote sudden accelerations and decelerations in the data set.

Other possible technical aspects for detecting the breaking points may be implemented.

The 2nd method allows detecting the ventilatory thresholds, during the session, by calculating in real-time the acceleration of the respiratory rate or, in another possible aspect, the change in the slope of the curve of the FR.

What is claimed is:

1. A portable device allowing measuring and characterising the fitness condition of a user in activity, characterised in that it includes:
At least one sensor measuring the respiratory rate and at least one second sensor measuring parameters of the activity of the user;
Means for calculating, with an accuracy equivalent to that of laboratory equipment, synthetic parameters that characterise the fitness including the V02max and for real-time and accurate detection of the ventilatory thresholds VT1, VT2, through measurement of the respiratory rate;
Means for characterising the activity of the user;
An interface for interaction with said user;
in that it includes several omnidirectional microphones, placed in an array according to the beamforming technique, with signal processing to create a directional microphone, which allows placing the device away from the mouth/nose;
and in that it includes redundant sensors to make the measurements more reliable.

2. The device according to claim 1, characterised in that it is autonomous.

3. The device according to claim 1, characterised in that it includes means for connecting to another device of the smartphone, smartwatch or digital tablet type, which are connected to a “Cloud”.

4. The device according to claim 1, characterised in that it includes means for directly connecting to a “Cloud”.

5. The device according to claim 1, characterised in that it includes means for association with an accessory, allowing clipping it or fastening it or integrating it into a piece of equipment, such as for example an earphone, a headphone or a headband, or inserting it into a piece of equipment.

6. The device according to claim 1, characterised in that it includes at least one microphone.

7. The device according to claim 1, characterised in that the at least one microphone is unidirectional.

8. The device according to claim 1, characterised in that it implements an algorithm for detecting audio rhythms or an artificial intelligence algorithm, for example a deep learning algorithm, of the microphone(s) in order to extract therefrom the respiratory rate and its intensity in a manner adapted to the activity, resistant to artefacts, in a noisy environment.

9. The device according to claim 1, characterised in that it includes an inertial platform, which includes a 3D accelerometer sensor, and a 3D gyroscope and/or a magnetometer and/or a barometer and/or a satellite positioning system.

10. The device according to claim 1, characterised in that it includes a microcontroller and/or an artificial intelligence processor for IoT devices, a memory, a BLE or other transmission mode module, a battery, one or more LED(s), a multifunction button, for activation, recording, transmitting and deleting the records, reset, or a plurality of buttons, a loudspeaker, a buzzer and/or vibrator, as well as other components of an IoT system.

11. The device according to claim 1, characterised in that it further includes a body parameter sensor and/or an environment parameter sensor.

12. The device according to claim 1, characterised in that it produces improved and adapted training instructions based on the perception of the effort and/or the ventilatory threshold.

13. The device according to claim 1, characterised in that it further includes means for detecting an alert.

14. The device according to claim 1, characterised in that it further includes means for analysing and characterising the breath and/or the cough via an artificial intelligence module.

15. The device according to claim 1, characterised in that it includes means for preventing the risk of fall and for performing exercises based on the feedback by training and monitoring a defective marker.

16. A system including a device according to claim 1, a device of the smartphone, smartwatch or digital tablet type, and a cloud-based server.

17. The system according to claim 16, characterised in that it further includes another device coupled with the device.
